(19) United States
(12) Patent Application Publication
(10) Pub. No.: US 2017/0156867 A1
(43) Pub. Date: Jun. 8, 2017

(54) Bone Graft Compositions and Methods for Making and Using the Same

(71) Applicant: Sahil Jalota, Carlsbad, CA (US)

(72) Inventor: Sahil Jalota, Carlsbad, CA (US)

(21) Appl. No.: 14/957,434

(22) Filed: Dec. 2, 2015

(51) Int. Cl. A61F 2/28; A61L 27/36

(52) U.S. Cl. CPC A61F 2/28; A61L 27/3608; A61L 27/3691; A61L 27/3687; A61L 2430/02

(57) ABSTRACT

Methods of producing bone graft materials are provided. Aspects of the methods include comminuting an initial bone source to produce a particulate bone composition, cleaning the particulate bone composition and then combining the cleaned particulate bone composition with an organic acid solution in a manner sufficient to produce a bone graft material. Aspects of the invention also include kits that find use in practicing the methods, as well as bone graft materials produced by the methods. The methods and bone graft materials produced thereby find use in a variety of applications, e.g., spinal fusion and bone void filling applications.

Bone grafting is a surgical procedure that places new bone or a replacement material into spaces between or around broken bone (fractures) or in holes in bone (defects) to aid in healing. Bone grafting is used to repair a variety of types of bone fractures, including those that are extremely complex, pose a significant risk to the patient, or fail to heal properly. Bone grafting is also used to help fusion between vertebrae, correct deformities, or provide structural support for fractures of the spine. In addition to fracture repair, bone grafting is used to repair defects in bone caused by congenital disorders, traumatic injury, or surgery for bone cancer. Bone grafts are also used for facial or cranial reconstruction.

The term “graft” commonly refers to an autograft or allograft. A graft made of bone from the patient's own body (e.g., hip bones or ribs) is an autograft. An allograft uses bone from another human source, such as a cadaver, where in some instances the bone has been frozen and stored in a tissue bank. Synthetic bone material may also be used as a graft.

To place a bone graft, a surgeon makes an incision in the skin over the bone defect, and shapes the bone graft or replacement material to fit into it. After the graft is placed into the defect, it may be held in place with pins, plates, or screws, as desired and/or necessary.

In surgery of the spine, especially spinal fusion (also called arthrodesis), surgeons may decide to use bone grafts to assist in the healing and remodeling of the spine after surgery. In such applications, small pieces of bone may be placed into the space between the vertebrae to be fused, and sometimes larger solid pieces of bone provide immediate structural support. Spinal fusion involves the surgical treatment of abnormalities in the vertebrae, such as curvatures, scoliosis or kyphosis, or injuries (fractures). For example, cervical spinal fusion joins selected bones in the neck. Bone grafts may be used for many different procedures, including but not limited to spinal fusion surgery, orthopedics, podiatry, dentistry, etc.

Demineralized Bone Matrix (DBM) is allograft bone that has had the inorganic mineral removed, leaving behind the organic collagen matrix. Removal of the bone mineral exposes more biologically active growth factors present in bone or bone marrow, e.g., bone morphogenetic proteins. These growth factors modulate the differentiation of progenitor cells into osteoprogenitor cells, which are responsible for bone and cartilage formation, thereby promoting bone growth. Bone tissue is a porous, matrix-like structure. The success of a bone graft is determined by its ability to recruit host cells to the site of the graft and modulate their conversion into bone forming cells.

### SUMMARY

Methods of producing bone graft materials are provided. Aspects of the methods include comminuting an initial bone source to produce a particulate bone composition, cleaning the particulate bone composition and then combining the cleaned particulate bone composition with an organic acid solution in a manner sufficient to produce a bone graft material. Aspects of the invention also include kits that find use in practicing the methods, as well as bone graft materials produced by the methods. The methods and bone graft materials produced thereby find use in a variety of applications, e.g., spinal fusion and bone void filling applications.

### DETAILED DESCRIPTION

Methods of producing bone graft materials are provided. Aspects of the methods include comminuting an initial bone source to produce a particulate bone composition, cleaning the particulate bone composition and then combining the cleaned particulate bone composition with an organic acid solution in a manner sufficient to produce a bone graft material. Aspects of the invention also include kits that find use in practicing the methods, as well as bone graft materials produced by the methods. The methods and bone graft materials produced thereby find use in a variety of applications, e.g., spinal fusion and bone void filling applications.

Before the methods of the present disclosure are described in greater detail, it is to be understood that the methods of the present disclosure are not limited to particular embodiments described, as such may, of course, vary. It is also to be understood that the terminology used herein is for the purpose of describing particular embodiments only, and is not intended to be limiting, since the scope of the methods of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges and are also encompassed within the present disclosure, subject to any specifically excluded limit in the stated range. Where the stated range includes one or both of the limits, ranges excluding either or both of those included limits are also included in the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which the present disclosure belongs. Although any methods and materials similar or equivalent to those described herein can also be used in the practice or testing of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

### Methods

As summarized above, methods of producing bone graft materials are provided. Aspects of the methods include comminuting an initial bone source to produce a particulate bone composition, followed by cleaning the particulate bone composition and then combining the cleaned particulate bone composition with an organic acid solution in a manner sufficient to produce a bone graft material. Each of these steps is now described in greater detail.

In producing bone graft materials of the invention, an initial bone source is comminuted to produce a particulate bone composition. By comminuted is meant to break into several small fragments, i.e., into a particulate composition. As such, comminuting produces a particulate bone fragment composition from an initial bone source. The ground particles can be of any shape or size desired. While the size of the particulates in the resultant particulate bone fragment composition may vary, in some instances the arithmetic mean of the particle diameters of the particles in a given composition ranges from 0.1 to 10 mm, such as 1 to 5 mm, including 1 to 4 mm. The initial bone source may be comminuted to produce the desired bone fragment composition using any convenient protocol, including but not limited to, grinding, pulverizing, etc.

The initial bone source that is comminuted to produce the particulate bone fragment composition may vary. Initial bone sources that may be employed in methods of the invention include autologous, allogeneic, or xenogeneic bone sources. Of interest in certain embodiments are allogeneic bone sources, e.g., mammalian bone sources, including primate bone sources, e.g., human bone sources. The initial bone source may be cortical bone, cancellous bone or a combination of cortical and cancellous bone. The initial bone source may be obtained from the shaft of long bones or from flat bone structures, and may or may not contain bone marrow. The initial bone source may include the outer periosteal layer, the middle layer or the inner endosteal layer or combinations thereof (e.g., derived from the periosteal layer and the middle layer) of a particular bone. The initial bone source may be raw bone, frozen bone or lyophilized (i.e., freeze dried) bone. In some instances, the initial bone source is cadaver bone, such as human cadaver bone. It is noted that in some embodiments, comminution as described above may be omitted from the process or done after cleaning and/or organic acid solution contact, e.g., as described below.

Following production, the resultant particulate bone fragment composition is cleaned. In some instances, cleaning the particulate bone fragment composition includes separating one of more of cell fragments, lipids and debris from the particulate bone composition. Cleaning may be performed using any convenient protocol. In some instances, cleaning includes contacting the bone fragments with a cleaning liquid under conditions sufficient to separate the one or more target components from the fragment composition. Any suitable cleaning liquid may be used for cleaning the bone fragments, wherein some instance cleaning results in defatting and/or disinfecting the bone fragments. The cleaning liquid employed in this step may vary and may be an aqueous composition. Examples of cleaning liquids include, but are not limited to: water, saline, peroxides, alcohols, crystalloids, sterilizing fluids, preserving fluids, storage agents, or other fluids used in processing of allografts. In some instances, the cleaning liquid is an aqueous liquid that includes one or more components, e.g., alcohols, such as ethanol, methanol, propanol, isopropanol, butanol; organic solvents, e.g., DMF, DMSO, diethyl ether, hexanes, dimethoxyethane, tetrahydrofuran, chloroform, methylene chloride and carbon tetrachloride; surfactants, e.g., Triton X-100, sterilants, e.g., hydrogen peroxide; and the like. Commercially available bone graft cleaning liquids may also be employed, e.g., Allowash XG® allograft cleaning liquid available from Lifenet.

Following cleaning of the particulate composition, the cleaned particulate bone composition is combined with an organic acid solution in a manner sufficient to produce the bone graft material. The organic acid solution employed in this step may vary, where the pH of the organic acid solution ranges, in some instances, from 0 to 7, such as 1 to 6. The concentration of the organic acid in the solution may vary, ranging in some instances from 0.1 to 70%, such as 1 to 50%, e.g., 5 to 45%. Of interest are organic acid solutions of a-hydroxy acid, e.g., glycolic acid, lactic acid, citric acid, mandelic acid. In some instances, the organic acid solution is a glycolic acid (i.e., hydroxyacetic acid) solution.

In some instances, the cleaned particulate bone composition is combined with the organic acid solution with mixing. Mixing may be accomplished using any convenient protocol, including by mechanical movements such as shaking, stirring, swirling, ultrasonicating, vibrating, vortexing, blending, grinding, or the like that causes mixing. Mixing of the components of the subject compositions can be carried out manually or by an automated mixer. Apparatuses for mixing of components to produce the subject compositions provided herein include for example, those described in U.S. Pat. Nos. 3,756,571; 3,828,434; 3,831,742; 3,917,062; 4,084,320; 4,551,135; 5,058,770, and 7,018,089, the disclosures of which are herein incorporated by reference.

### Bone Graft Materials

Aspects of the invention further include bone graft materials produced by methods of the invention. The bone graft materials are characterized by including a collagen component and a calcium phosphate component. The collagen component is made up of collagen that is present in the initial bone source. The calcium phosphate component includes, in some instances, a calcium phosphate mineral that is not present in the initial bone source, e.g., a calcium phosphate mineral that is produced by reaction of calcium phosphate molecules in the initial bone source and the organic acid, e.g., glycolic acid. The reaction between the calcium phosphate mineral of the bone and organic acid solution, e.g., glycolic acid solutions, produces a product, e.g., having a putty like consistency that can be used as an impactable form or can be injected through a syringe with or without cannula. The reaction of calcium phosphate mineral present in bone (which typically includes hydroxyapatite) with the organic acid solution can form one or more calcium phosphate minerals that are distinct from that found in the original bone, e.g., monocalcium phosphate (anhydrous or monohydrate), dicalcium phosphate (anhydrous (monetite or dihydrate (brushite), octacalcium phosphate. \The reaction is characterized by stripping the original calcium phosphate mineral phase from the collagen surface of the bone and formation of the aforementioned calcium phosphate(s) in a gel matrix incorporates the original collagen component. Removal of the original bone mineral from the collagen surface exposes more biologically active factors, e.g., bone morphogenetic proteins.

The bone graft materials may be osteoconductive and/osteoinductive. As used herein “osteoconductive” refers to the ability to provide a scaffold for which bone cells can attach to initiate bone growth. As used herein, “osteoinductive” refers to the ability to induce bone growth. In certain instances, the subject bone graft materials are bioresorbable. Bioresorbable compositions of the invention can be broken down and assimilated in a subject (e.g., a human subject) after a period of time. As such, bioresorbable products allow for a temporary scaffold for tissue growth (e.g., hard tissue) or for the delivery of an agent in a subject (e.g., an agent that promotes bone growth). In certain instances, the bone graft compositions remain in a subject for 1, 2, 3, 4, 5, 6, 7, 8, 9, 10, 11, 12 weeks or less following implantation. In certain instances, the bone graft compositions remain in a subject for longer period of time, e.g., 6 months, 1 year, 2 years, 5 years or longer.

Where desired, the bone graft materials may include one or more osteoinductive factors. Osteoinductive factors that may be present in the compositions include, but are not limited to: bone morphogenetic growth factors (BMPs, e.g., BMP-2 and BMP-7), IGF, TGF-β, IGFs (IGF-1 and IGF-2), parathyroid hormone, angiogenic factors (VEGF, aFGF, bFGF), mitogenic factors, osteocalcin and osteopontin.

In some instances, the bone graft materials include one or more agents that aid in the repair of a bone defect. In certain instances, the agent(s) present in the composition is an agent not found in naturally occurring bone. Exemplary agents include, but are not limited to, antiviral drugs, antibiotics, antimicrobial drugs, growth factors (e.g., osteoinductive or osteogenic factors), and immunosuppresants.

In certain embodiments, the bone graft materials include are radiographic. As used herein, the term “radiographic” refers to the ability to be visualized by radiography. As such, the compositions may be visualized by X-ray radiography following implantation in the subject, e.g., by standard radiography techniques. In certain embodiments, the radiodensity of the graft composition ranges from +30 to +5000 Hounsfield units (HU), including between +100 to +3,000 HU. As used herein, “radiodensity” refers to the relatively inability of electromagnetic radiation (e.g., X-ray) to pass through a particular material, as measured in HU.

In some instances, the bone graft materials are flowable compositions. The term “flowable” is meant to include putty and paste-like compositions, as well as more liquid compositions. “Flowable” as used herein therefore refers to both injectable and compactable compositions. In certain embodiments, the viscosity time of the flowable compositions, defined as time periods under which the mixed composition injects through a standard Luer-lok fitting after mixing, ranges up to 10 minutes or longer, such as up to about 7 minutes or longer, and including up to about 4 minutes or longer. Of interest in certain embodiments are paste compositions that have an injectable viscosity that injects in a time period ranging up to about 5 minutes, such as about up to about 4 minutes.

Where desired, the bone graft material produced, e.g., as described above, may be sterilized. In various embodiments, the bone graft material is sterilized by radiation in a sterilization step, e.g., before and/or after it is placed into any desired packaging, such as sterile packaging. In various embodiments, gamma radiation is used in the terminal sterilization step, which involves utilizing ionizing energy from gamma rays that penetrates deeply in material. Gamma rays are highly effective in killing microorganisms, they leave no residues nor have sufficient energy to impart radioactivity to the device. Gamma rays can be employed when the material is present a package and gamma sterilization does not require high pressures or vacuum conditions, thus, package seals and other components are not stressed. In addition, gamma radiation eliminates the need for permeable packaging materials. In various embodiments, electron beam (e-beam) radiation may be used to sterilize the material. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. Other methods may also be used to sterilize the device and/or one or more components of the device, including, but not limited to, gas sterilization, such as, for example, with ethylene oxide or steam sterilization.

### Utility

The bone graft materials, e.g., as described above, find use in a variety of applications. The bone graft materials, e.g., as described above, are useful, for example, in instances where bone growth is desired, for example, for repair of a bone defect, for bone replacement, for bone augmentation, etc. The bone graft materials, e.g., as described above, find use in applications where it is desired to introduce an implantable composition having osteoconductive, osteoinductive and/or osteogenic properties into a physiological site of interest, such as in orthopedic applications.

In orthopedic applications, the bone grafter material may be prepared, as described above, and introduced to a site where bone growth is desired (e.g., bone repair site), including sites containing cancellous and/or cortical bone. In certain embodiments, the bone graft materials are used for the treatment of a subject having a bone disease or disorder. The bone graft materials can be applied at a desired physiological site using any suitable applicator, e.g., syringe, cannula, catheter, spatula, etc., depending on the viscosity of the composition.

Subjects that can be treated using the subject composition include mammalian subjects, e.g., primates, including humans. In some embodiments, the bone graft materials are used for the treatment of a bone fracture, bone cancer, bone metastases, an autoimmune disease, a metabolic disease, rheumatoid arthritis, or a degenerative bone disease. In some instances, the bone graft materials are used for the repair of a fracture. In other instances, the bone graft materials are used for implant augmentation. In yet other instances, the bone graft materials are used for the replacement of a bone.

Fractures that can be treated with the subject bone graft materials include simple fractures, compound fractures and non-unions. In such fracture treatment methodologies, the fracture is first reduced. Following fracture reduction, a subject composition is introduced into the cancellous tissue in the fracture region using an application (e.g., a spatula or cannula). Bones that are suitable for repair and replacement using the subject compositions include, but are not limited to: calcaneus, carpal, cervical vertebra, clavicle, ethmoid, femur, fibula, frontal, humerus, ilium, ischium, lumbar vertebra, mandible, maxilla, metacarpal, metatarsal, nasal, occipital, parietal, patella, phalanges, pubis, radius, rib, sacrum, scapula, sternum, tarsal, temporal, thoracic vertebra, tibia, ulna, and zygomatic bones.

Bone graft materials of the invention are useful in spinal applications including restoration of column support. The bone graft materials are useful for implantation inpatients suffering from defects caused by congenital anomaly, disease, or trauma, including for example, spine fractures; deformity, e.g., kyphotic deformities, e.g., posttraumatic kyphosis; postlaminectomy kyphosis, junctional kyphosis, and Scheuermann's kyphosis; scoliosis, e.g., neuromuscular scoliosis, adult scoliosis, paralytic scoliosis, congenital and syndromic scoliosis; and cervical neck pain. Surgical methods for correcting degenerative conditions, for example in the lumbar spine, include decompression (excision of disc material, hypertrophied bone, or ligament) along with fusion, or fusion alone.

Where desired, a posterior surgical approach may be employed. The choice of approach is dictated by the site of primary pathology. Pathology that involves vertebral bodies may be approached anteriorly through the thorax, abdomen or flank. Pathology involving posterior elements are best approached posteriorly for example, through a vertical midline approach or posterior lateral muscle spinning approach.

Those of ordinary skill in the art to which the present invention pertain, including for example an orthopedic surgeon and a spinal surgeon, can readily select and employ a particular bone graft material of the invention, without undue experimentation. An ideal graft, for example for use in lumbar interbody fusion, should be: osteoinductive, non-immunogenic, provide immediate mechanical stability, and be appropriately sized and shaped for the particular application/patient. Indications, diagnostic criteria, graft selection and surgical technique, are factors that can be readily selected, optimized and employed by those of ordinary skill in the art without undue experimentation, and are discussed in: Master Techniques in Orthopaedic Surgery, The Spine, edited by Bradford, David S., Lippincott-Raven, ISBN 0-7817-0033-7, Philadelphia, Pa., (1997), hereby incorporated herein by reference in its entirety. When implanting a cervical fusion graft, an anterior cervical approach may be employed.

Where desired, compositions that are implanted in a subject may be imaged, e.g., using any suitable radiography technique to obtain an image of the implanted composition. In certain instances, images of the composition are produced by passing a radiation source (e.g., an X-ray generator) over a subject at the site where the composition has been implanted and capturing the radiation that passes through the subject onto a recording medium. In certain embodiments, the recording medium is film. In other embodiments, the recording medium is a digital recording medium (e.g. a digital detector). In some embodiments, the X-ray generator and recording medium is capable of measuring bone mineral density at the site of implantation of the subject composition. Exemplary systems for obtaining X-ray images are described in U.S. Pat. Nos. 5,204,888; 6,285,740; 6,320,931; 6,325,537; 6,666,579; and 7,672,432, the disclosure of which is herein incorporated by reference.

### Kits

Also provided are kits that include a bone graft material, e.g., as described above. In addition to the bone graft materials, the subject kits may further include a number of additional components, e.g., applicators, such as described above. The component(s) of the kit may be present in sterile packaging, e.g., a sterile pouch, as desired. The various components may be present in the same or different containers, e.g., sterile packaging.

In addition to above-mentioned components, the subject kits may include instructions for using the components of the kit to practice the subject methods. The instructional material may also be instructional material for using the bone graft materials, e.g., it may provide surgical techniques and protocols for a particular application in which the bone graft material is to be employed. The instructions for practicing the subject methods may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., portable flash drive, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

I. Bone Graft Putty Preparation
A. Comminuting

Human cadaver long bone (obtained from long bone shaft) is ground into fragments having a diameter of 4mm particles using a bone bill or grinder.
B. Cleaning

The resultant ground human bone fragments are cleaned using following process:

a. Stirring/shaking in 1% Triton X-100 solution for 30-90 minutes

b. Rinsing with PBS for 5 minutes

c. Stirring/shaking with 3% Hydrogen Peroxide for 180-240 minutes

d. Rinsing with PBS for 5 minutes
C. Formation of Bone Putty Graft Material

Mixing with glycolic acid: 3 grams of cleaned bone fragments are mixed with dilute glycolic acid (3 grams water+3 grams concentrated glycolic acid). The mixture is blended in a blender for 2 minutes and a homogeneous bone putty is formed.
D. Bone Putty Graft Material Characteristics

The resultant bone putty exhibits osteoinductive and osteoconductive properties. The putty is radio-opaque (in contrast to other demineralized bone material (DBM) grafts which are radio-lucent) and can be injected via a 8- or 11-gauge cannula. The material has greater than 1000 ng/g BMP-2 as tested using in vitro ELISA techniques.
II. Bone Putty Graft Material Induces Spinal Fusion in RAT PLF Model
A. Surgery: All animal procedures and surgeries are performed under an approved IACUC protocol at a contract laboratory. Four animals are used for bilateral implantation of 0.3 cc graft material, as prepared in Example I above, in between the L4 and L5 transverse processes. Two Animals are euthanized at each six (6) and twelve (12) weeks post-surgery. Samples are excised one level above and below the fusion mass, and analyzed for fusion by manual palpation, radiographs, microCT and histology.
B. Manual Palpation: After trimming the soft tissue from the spine segments, each spine segment is independently evaluated by three observers. A three point scoring system (0: not fused; 1: unilateral fusion; 2: bilateral fusion) is used, and observations noted. After evaluation, the spinal segments are placed in 10% neutral buffered formalin for fixation.
C. Radiography: One Faxitron radiograph is taken for each specimen and graded (0: not fused; 1: unilateral fusion; 2: bilateral fusion) twice by a single observer for fusion. The two scores are averaged, and rounded down to obtain a single fusion score per animal.
D. MicroCT: Each side of the sample is scored (0: not fused; 1: fusion), and added together to get a fusion score per animal.
E. Histology: Each fusion site was isolated and trimmed of soft tissue, decalcified, and embedded in paraffin. Three step sections were taken from each block, hemotoxylin and eosin stained, and analyzed.
F. Results:

Fusion is observed in all test animals, with 1 out of 2 demonstrating bilateral fusion at six weeks and both animals (2) demonstrating bilateral fusion at twelve weeks.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of producing a bone graft material, the method comprising:
comminuting an initial bone source to produce a particulate bone composition;
cleaning the particulate bone composition; and
combining the cleaned particulate bone composition with an organic acid solution in a manner sufficient to produce the bone graft material.

2. The method according to claim 1, wherein the initial bone source comprises raw bone.

3. The method according to claim 1, wherein the initial bone source comprises freeze-dried bone.

4. The method according to claim 1, wherein the initial bone source comprises frozen bone.

5. The method according to claim 1, wherein the initial bone source comprises cadaver bone.

6. The method according to claim 1, wherein the comminuting comprises grinding.

7. The method according to claim 1, wherein the cleaning comprises separating one of more of cell fragments, lipids and debris from the particulate bone composition.

8. The method according to claim 7, wherein the cleaning comprises contacting the bone particulate composition with a cleaning liquid.

9. The method according to claim 8, wherein the cleaning liquid comprises a surfactant.

10. The method according to claim 8, wherein the cleaning liquid comprises a sterilant.

11. The method according to claim 1, wherein the organic acid solution has a pH ranging from 0 to 7.

12. The method according to claim 1, wherein the organic acid solution comprises an organic acid at a concentration ranging from 1 to 70%.

13. The method according to claim 1, wherein the organic acid solution comprises an a-hydroxy acid.

14. The method according to claim 13, wherein the a-hydroxy acid is glycolic acid.

15. The method according to claim 1, wherein the cleaned particulate bone composition is combined with the organic acid solution with mixing.

16. The method according to claim 1, wherein the method further comprises harvesting the initial bone source.

17. The method according to claim 1, wherein the initial bone source comprises human bone.

18. The method according to claim 1, wherein the method further comprises sterilizing the bone graft material.

19. A bone graft material produced according to the method of claim 1.

20-23. (canceled)

24. A method comprising implanting a bone graft material according to claim 19 in a subject.

25-32. (canceled)
